(19) United States
(12) Patent Application Publication
(10) Pub. No.: US 2022/0372535 A1
(43) Pub. Date: Nov. 24, 2022

(54) FRUCTOSE-6-PHOSPHATE 3-EPIMERASE AND USE THEREOF

(71) Applicant: SAMYANG CORPORATION, Seoul (KR)

(72) Inventors: Jinsol HEO, Seongnam-si (KR); In-Suk JOUNG, Yongin-si (KR); Eunsoo CHOI, Seongnam-si (KR)

(21) Appl. No.: 17/772,222

(22) PCT Filed: Oct. 28, 2020
(86) PCT No.: PCT/KR2020/014808

§ 371 Date: Apr. 27, 2022

(30) Foreign Application Priority Data
Oct. 28, 2019 (KR) 10-2019-0134730
Oct. 28, 2020 (KR) 10-2020-0141069

(51) Int. Cl. C12P 19/02; C12P 19/24; C12N 9/90

(52) U.S. Cl. CPC C12P 19/02; C12P 19/24; C12N 9/90; C12Y 501/03

(57) ABSTRACT

The present disclosure relates to an epimerase protein of fructose-6-phosphate, nucleic acid molecule encoding the epimerase protein, a recombinant vector and a transgenic microorganism which comprise the nucleic acid molecule, and a composition for producing allulose by using them.

### TECHNICAL FIELD

The present disclosure relates to an epimerase protein for phosphorylated saccharide, and more particularly to a fructose-6-phosphate epimerase protein, a nucleic acid molecule for encoding the enzyme protein, a recombinant vector and a transgenic strain which comprise the nucleic acid molecule, and a composition for producing allulose using the strain.

### BACKGROUND ART

Phosphorylated saccharide epimerase is an epimerase bound to the carbon of various phosphorylated saccharides, and ketohexose-6-phosphate epimerase can epimerize C3 or C4. The ketohexose may be one or more ketohexoses selected from the group consisting of fructose, allulose, sorbose, and tagatose.

Fructose-6-phosphate epimerases include 3-epimerase and 4-epimerase. Specifically, D-allulose-3-epimerase (EC 5.1.3.30) produces allulose-6-phosphate through 3-epimerization (epimerization at the C-3 position) of fructose (D-fructose).

When producing allulose from fructose using the above enzyme, a certain level of reaction equilibrium exists between fructose used as a substrate and allulose as a product, so that the final conversion rate is only 20 to 35%. Therefore, when attempting to prepare high-purity allulose through a single reaction using the enzyme, an additional process of separating and removing fructose from the final reaction solution is required.

When industrially attempting to produce allulose from fructose as a raw material, the enzyme used must have high industrial production conditions, especially thermal stability, and the highest possible conversion rate. Further, saccharides are used as a substrate and browning of saccharides occurs easily under alkaline conditions, it is necessary to satisfy the conversion reaction condition to prevent browning of sugar as much as possible.

Therefore, there is an urgent need for an enzyme that satisfies at least one of u substrate conversion rate, thermal stability of the enzyme, and enzyme reaction conditions being suitable for industrial use, and that produces phosphorylated fructose using fructose as a raw material, and a method for producing phosphorylated fructose using the same.

### DETAILED DESCRIPTION OF THE INVENTION

### Technical Problem

One aspect of the present disclosure relates to a fructose-6-phosphate 3-epimerase protein, a nucleic acid molecule for encoding the enzyme protein, a recombinant vector and a transgenic microorganism which comprise the nucleic acid molecule.

Another aspect of the present disclosure relates to a composition and a method for producing an allulose-6-phosphate using fructose-6-phosphate and a composition for producing an allulose-6-phosphate using fructose-6-phosphate, comprising at least one selected from the group consisting fructose-6-phosphate 3-epimerase protein, a microbial cell of microorganism expressing the enzyme, a cell lysate of the microbial cell, a culture of the microorganism, a culture supernatant of the microorganism or their extracts.

Further aspect of the present disclosure relates to a composition and a method for producing ketohexose such as allulose using fructose-6-phosphate, comprising at least one selected from the group consisting of a fructose-6-phosphate 3-epimerase protein, a microbial cell expressing the enzyme, a cell lysate of the microbial cell, a culture of the microorganism, a culture supernatant of the microorganism or their extracts.

### Technical Solution

In order to achieve the above objects, one embodiment of the present disclosure relates to fructose-6-phosphate 3-epimerase protein comprising an amino acid sequence that has 70% or more, 80% or more, 90% or more, 95% or more, 97% or more, or 99% or more sequence homology to the amino acid sequence of SEQ ID NO: 1. For example, it is obvious that any protein having an amino acid sequence with deletion, modification, substitution, or addition in part of the sequence may also be included within the scope of the present disclosure, as long as the amino acid sequence has the homology described above and exhibits the efficacy corresponding to the protein consisting of the amino acid sequence of SEQ ID NO: 1.

An embodiment of the present disclosure provides a fructose-6-phosphate 3-epimerase comprising an amino acid sequence that has 70% or more, 80% or more, 90% or more, 95% or more, 97% or more, or 99% or more sequence homology to the amino acid sequence of SEQ NO: 1. The fructose-6-phosphate 3-epimerase may he encoded by the nucleotide sequence of SEQ ID NO: :2, or a nucleotide sequence having at least 80%, 90%, 95%, 97%, or 99% or more homology to the nucleotide sequence of SEQ ID NO: 2.

As an amino acid sequence showing the homology of the above numerical values, any fructose-6-phosphate 3-epimerase protein substantially identical to or corresponding to the enzyme may be included without limitation. Further, as long as the sequence having such homology is an amino acid sequence that substantially exhibits fructose-6-phosphate 3-epimerase function, protein variants with deletion, modification, substitution, or addition in part of the sequence are also included within the scope of the present disclosure.

As used herein, the term “homology” or “identity” refers to a degree of matching with a given amino acid sequence or nucleotide sequence, and it may be expressed as a percentage. In the present disclosure, a homology sequence having activity which is identical or similar to the given amino acid sequence or nucleotide sequence is expressed as “% homology”.

The fructose-6-phosphate 3-epimerase according to the present disclosure catalyzes the 3-epimerization reaction of fructose 6-phosphate, and specifically, may perform 3-epimerization of fructose-6-phosphate convened to allulose-6-phosphate.

The fructose-6-phosphate 3-epimerase protein according to the present disclosure may be an enzyme derived from Clostridium lundense, and specifically, an enzyme derived from Clostridium lundense DSM 17049.

The reaction temperature range of the Clostridium lundense-derived fructose-6-phosphate 3-epimerase may be 40 to 70° C., 45 to 75° C., 45 to 77° C., 50 to 70° C., or 50 to 75° C. The optimum temperature may be, for example, the result of the reaction proceeding for 5 minutes under the condition of pH 7.0, but is not limited thereto. The optimum temperature condition for fructose-6-phosphate 3-epimerase is 60° C., and it has an activity of 50% or more of the maximum enzyme activity in a wide temperature range of 40 to 70° C.

The reaction pH range of the Clostridium lundense-derived fructose-6-phosphate 3-epimerase may be pH 6 to 8, pH 6 to 7.5, pH 6.5 to 8, pH 6.5 to 7.5, pH 7 to 8, or pH 7 to 7.5, and it has the maximum activity under the condition of pH 7.0 to 7.5, and has an activity of 80% or more of the maximum enzyme activity in the range of pH 6.0 to 8.0.

The maximum allulose production amount of the fructose-6-phosphate 3-epimerase derived from Clostridium lundense is 16 wt % or more, 18 wt % or more, 20 wt % or more, :25 wt % or more, 27 wt % or more, 30 wt % or more or 32% by weight or more. Specifically, the maximum allulose production amount of the enzyme may be measured for a reaction performed by adding 0.1 mg/ml. of the enzyme to a dissolved solution of 20 g/L fructose-6-phosphate, and more specifically, may be measured by adding 0.1 mg/ml of the enzyme to a dissolved solution of 20 g/L fructose-6-phosphate an enzymatic reaction at pH 7.0 and 50° C.

The maximum conversion rate of allulose may be calculated by Equation 1 below. The time of the enzymatic reaction to obtain the maximum conversion rate of allulose may be 8 hours or more, 10 hours or more, 12 hours or more, 14 hours or more, or 16 hours or more, and the upper limit of the reaction time may be 18 hours or less or 20 hours or less. The specific reaction time may be a range combining the lower limit and the upper limit, for example, may be 16 hours to 70 hours.

### Equation 1

Allulose maximum conversion rate (%)=(allulose production amount (g/L)/fructose-6-phosphate input amount (g/L)*(allulose molecular weight/fructose-6-phosphate molecular weight)*100

The ratio (% by weight) of allulose production of the saccharides in the enzymatic reaction product can be calculated by the following equation. The enzyme reaction time for obtaining the allulose production rate may be 2 hours to 6 hours, 3 hours to 6 hours, or 4 hours to 6 hours, for example, 4 hours to 6 hours.

### Equation 2

Allulose production ratio (% by weight)=allulose production amount/(fructose production amount+allulose production amount)*100

The fructose-6-phosphate 3-epimerase protein according to the present disclosure may be increased or decreased in its enzyme activity by metal ions. Specifically, the enzyme activity of the fructose-6-phosphate 3-epimerase protein is increased by Mn, Co and Ni ions. For example, it exhibits an activity as 1.1 times or more, 1.2 times or more, 1.3 times or more, or 1.5 times or more as compared with the condition without metal ions. In particular, Mn and Co ions exhibit an activity of 2 dines or more, or 3 times or more, specifically 2 to 5 times, 2 to 4 times, 3 to 5 times, or 3 to 5 times as compared with the condition without metal ions. Fructose-6-phosphate 3-epimerase protein has a property that its activity is reduced by Ca, Cu, Fe, or Zn ions.

The fructose-6-phosphate 3-epimerase according to an embodiment of the present disclosure has 70% or more, 80% or more, 90% or more, 95% or more, 97% or more, or 99% or more sequence homology to the amino acid sequence of SEQ ID NO: 1. It is obvious that any protein having an amino acid sequence with deletion, modification, substitution, or addition in part of the sequence may also be included within the scope of the present disclosure, as long as the amino acid sequence exhibits the efficacy corresponding to the protein consisting of the amino acid sequence of SEQ ID NO: 1.

For the Clostridium lundense-deli ved fructose-6-phosphate 3-epimerase protein, the amino acid sequence identity to Ruminococcus sp. AF14-10-derived ribulose-phosphate 3-epimerase (RuFP3E: amino acid sequence of SEQ ID NO: 5), Clostridium sp. DL-VIII-derived ribulose-phosphate 3-epimerase (CDFP3E: amino acid sequence of SEQ ID NO: 7), and Paenibacillus kribbensis-derived ribulose-phosphate 3-epimerase (PkFP3E: amino acid sequence of SEQ ID NO: 9) was analyzed, and as a result, the sequence identity to RuFP3E was 59.05%, the sequence identity to CDFP3E was 63%, the amino acid sequence identity to PkFP3E was 60.96%, and the sequence homology to the amino acid sequence of SEQ ID NO: 1 was less than 70%.

Further, as a result of HI′LC analysis of the conversion activity of fructose 6-phosphate to allulose 6-phosphate, an allulose peak was indentified in the ClFP3E enzyme having SEQ ID NO: 1, but an allulose peak was not identified in the RuFP3E, CDFP3E and PkFP3E. Thus, because other candidate enzymes other than the ClFP3E enzyme had no activity against F6P, it can he confirmed that when phosphatase is treated, only fructose in the form in which phosphate group is removed from F6P, which is the initial substrate of the reaction step, is generated (FIG. 7a, 7b).

A further embodiment of the present disclosure provides a nucleic acid molecule encoding the fructose-6-phosphate 3-epimerase of the present disclosure. In a specific embodiment of a nucleic acid encoding a fructose-6-phosphate 3-epimerase according to the present disclosure, the fructose-6-phosphate 3-epimerase may include the nucleotide sequence of SEQ ID NO: 2 or a nucleotide sequence of at least 80% or more, 90% or more, 95% or more, 97% or more, or 99% or more homology to the nucleotide sequence of SEQ ID NO: 2.

Yet another embodiment of the present disclosure provides a vector or transformant comprising a nucleic acid encoding the fructose-6-phosphate 3-epimerase of the present disclosure.

As used herein, the term “transformation” refers to a process of introducing into a host cell a vector including a nucleic acid encoding a target protein, thereby enabling the expression of the protein encoded by the nucleic acid in the host cell. For the transformed nucleic acid, it does not matter whether the transformed nucleic acid is inserted into the chromosome of a host cell and located therein or located outside the chromosome, as long as it can be expressed in the host cell, and both cases are included. Additionally, the nucleic acid includes DNA and RNA which encode the target protein. The nucleic acid may be inserted in any form as long as it can be introduced into a host cell and expressed therein. For example, the nucleic acid may be introduced into a host cell in the form of an expression cassette, which is a gene construct including all essential elements required for self-expression. The expression cassette may conventionally include a promoter operably linked to the nucleic acid, a transcription termination signal, a ribosome-binding domain, and a translation termination signal. The expression cassette may be in the form of an expression vector capable of self-replication. Additionally, the nucleic acid may be introduced into a host cell as it is and operably linked to a sequence essential for its expression in the host cell, but the nucleic acid is not limited thereto.

Additionally, as used herein, the term “operably linked” refers to a functional linkage between a promoter sequence, which initiates and mediates the transcription of the nucleic acid encoding the target protein of the present disclosure, and the above gene sequence.

The method of the present disclosure for transforming the vector includes any method of introducing a nucleic acid into a cell, and may be carried out by selecting a suitable standard technique known in the art according to a host cell. Examples of the method may include electroporation, calcium phosphate (CaPO₄) precipitation, calcium chloride (CaCl₂) precipitation, microinjection, a polyethyleneglycol (PEG) technique, a DEAE-dextran technique, a cationic liposome technique, a lithium acetate-DMSO technique, etc., but are not limited thereto.

As the host cell, it is preferable to use a host having a high efficiency of introducing DNA and a high efficiency of expressing the introduced DNA. For example, it may be E. coli, but is not limited thereto.

Yet another embodiment of the present disclosure provides a composition for producing ketohexose, for example, allulose, comprising at least one selected from the group consisting of the fructose-6-phosphate 3-epimerase according to the present disclosure, a microorganism expressing the enzyme protein, a transgenic microorganism expressing the enzyme protein, a microbial cell of the microorganism, a cell lysate of the microbial cell of the microorganism, a culture of the microorganism, a culture supernatant of the microorganism, a concentrate of the culture supernatant of the microorganisms and their powders.

The culture contains an enzyme produced from a microorganism that produces fructose-6-phosphate 3-epimerase, and may be in a cell-free form with or without the strain. The cell lysate means a lysate obtained by disrupting the microbial cells of a microorganism producing fructose-6-phosphate 3-epimerase or a supernatant obtained by centrifuging the lysate, and contains an enzyme produced from a microorganism that produces the polyphosphate-dependent glucose kinase.

The culture of the strain contains the enzyme produced from the microorganism producing the fructose-6-phosphate 3-epimerase, and may he in a cell-free form with or without the microbial cells. Unless otherwise stated herein, the microorganisms used to produce fructose-6-phosphate 3-epimerase means inclusion of at least one selected from the group consisting of the microbial cell of the strain, the culture of the strain, the lysate of the microbial cell, the supernatant of the lysate, and extracts thereof.

The method for producing ketohexose, for example, allulose, according to the present disclosure is environmentally friendly, because it uses an enzyme obtained from a microorganism. The conversion of allulose from fructose by using a simple enzymatic reaction of an new method, significantly reduces production costs and maximizes the production effect.

The composition for producing allulose according to the present disclosure may further include one or more metal ions selected from the group consisting of Mn, Co, and Ni ions. The concentration of the metal ion may be 0.5 mM to 20 mM, and for example, it may be 0.5 mM to 10 mM, 1.0 mM to 10 mM, 1.5 mM to 8.0 mM, 2.0 mM to 8.0 mM, 3.0 mM to 7.0 mM, 4.0 mM to 6.0 mM, or 0.2 mM to 10 mM.

When producing ketohexose allulose using the composition for producing allulose, the reaction temperature and reaction pH conditions using the enzyme or the enzyme-producing microorganism are the same as described above in the reaction temperature and reaction pH conditions of the enzyme.

The composition for producing allulose may include at least one selected from the group consisting of hexokinase enzyme converting fructose to fructose-6-phosphate, a transgenic microorganism expressing the enzyme protein, a microbial cell of the microorganism, a microbial cell lysate of the microorganism, a culture of the microorganism, a culture supernatant of the microorganism, a concentrate of the culture supernatant of the microorganisms and their powder.

The fructose,-6-phosphate is preferably obtained by hexokinase treatment of fructose or a fructose-containing material, but also falls within the scope of the present disclosure, even if it is provided by other chemical synthesis method or the like.

The fructose-6-phosphate may be prepared from glucose-6-phosphate, and the composition for producing allulose may further include a glucose-6-phosphate isomerase converting glucose-6-phosphate to fructose-6-phosphate by isomerizing glucose-6-phosphate.

The glucose-6-phosphate can be prepared by direct phosphorylation of glucose, or may be converted from glucose-1-phosphate. Glucose may be obtained by treating starch or starch hydrolyzate, for example, dextrin and the like with gluconeogenesis amylase, and glucose-1-phosphate can be obtained by treating the glucose with a phosphorylation enzyme. The composition for producing ketohexose may further include an enzyme system for producing glucose-6-phosphate.

Specifically, the enzyme contained in the composition of the present disclosure for producing allulose and the substrate used for producing allulose are not limited. The composition of the present disclosure for producing allulose may further include (a) (i) starch, maltodextrin, sucrose, or a combination thereof; (ii) phosphate; (iii) allulose-6-phosphate phosphatase; (iv) glucose-6-phosphate-isomerase; (v) phosphoglucomutase or glucose phosphorylase; and (vi) α-glucanophosphorylase, starch phosphorylase, maltodextrin phosphorylase, sucrose phosphorylase, α-amylase, pullulanase, isoamylase, glucoamylase or sucrase; or (b) a microorganism expressing any of the enzymes described in item (a) or a culture of the microorganism, but it not limited thereto.

Specifically, the starch/maltodextrin phosphorylase (EC 2.4.1.1) and α-glucanophosphorylase of the present disclosure ay include any proteins as long as these are proteins that are subjected to phosphoryl transfer hosphate to glucose, thereby having the activity of producing glucose-1-phosphate from starch or maltodextrin.

The sucrose phosphorylase (EC 2.4.1.7) of the present disclosure may include any protein as long as it is a protein that is subjected to transfer phosphate to glucose, thereby having the activity of producing glucose-1-phosphate from sucrose.

The α-amylase (EC 3,2,1.1), pullulanase (EC 32141), glucoamylase (EC 321,3), and isoamylase of the present disclosure, which are enzymes for starch saccharification, may include any proteins as long as these are proteins having the activity of converting starch or maltodextrin to glucose.

The sucrase (EC 3,2126) of the present disclosure may include any protein as long as it is a protein having the activity of converting sucrose to glucose. The phosphoglucomutase (EC 5.4.2.2) of the present disclosure may include any protein as long as it is a protein having the activity of converting glucose-1-phosphate, to glucose-6-phosphate. The glucokinase may include any protein as long as it is a protein capable of transferring phosphate to glucose, thereby having the activity of converting to glucose,-6-phosphate. Specifically, the glucokinase may be a polyphosphate-dependent glucokinase.

The glucose-6-phosphate isomerase of the present disclosure may include any protein as long as it has an activity to convert glucose-6-phosphate to fructose-6-phosphate.

The allulose-6-phosphate dephosphorelyation enzyme of the present disclosure may include any protein as long as it is a protein having the activity of converting allulose-6-phosphate to allulose. More specifically, the allulose-6-phosphate phosphatase may be a protein having an activity of irreversibly converting allulose-6-phosphate to allulose. The composition for producing allulose may further include phytase, which performs a dephosphorylation reaction in allulose-6-phosphate, for example, allulose-6-phosphate phosphatase, but is not limited thereto. The composition for producing ketohexose may further include allulose-6-phosphate phosphatase, microorganisms expressing the allulose-6-phosphate phosphatase or a culture of a microorganism expressing the allulose-6-phosphate phosphatase.

The reaction temperature and reaction pH conditions using enzymes for producing ketohexose, for example, allulose-6-phosphate using the composition for producing ketohexose, or microorganisms that produce enzymes are the same as described above for the reaction temperature and reaction pH conditions of the enzymes.

The method may further include preparing fructose-6-phosphate from fructose or a fructose-containing material using hexokinase, and/or may further include contacting phosphatase with a microorganism expressing the same, or a culture of the microorganism to remove the phosphate.

The step of removing the phosphate can perform using allulose-6-phosphate phosphatase, a microorganism expressing the allulose-6-phosphate phosphatase or a culture of a microorganism expressing the allulose-6-phosphate phosphatase to produce allulose. The allulose 6-phosphate can remove a phosphate group by other enzymes or chemical methods.

An embodiment of the present disclosure provides a method for producing allulose, which further comprises contacting a fructose-6-phosphate3-epimerase, a microorganism expressing the fructose-6-phosphate 3-epimerase, or a culture of a microorganism expressing the fructose-6-phosphate 3-epimerase, thereby converting fructose-6-phosphate to allulose-6-phosphate.

After the step of converting fructose-6-phosphate of the present disclosure to allulose-6-phosphate, the preparation method of the present disclosure may further include a step of contacting allulose-6-phosphate with allulose-6-phosphate phosphatase, a microorganism expressing the allulose-6-phosphate phosphatase, or a culture of a microorganism expressing the allulose-6-phosphate phosphatase, thereby converting the allulose-6-phosphate to allulose.

Before the step of converting fructose-6-phosphate of the present disclosure to allulose-6-phosphate, the preparation method of the present disclosure may further include a step of contacting glucose-6-phosphate with glucose-6-phosphate-isomerase, a microorganism expressing the glucose-6-phosphate-isomerase, or a culture of a microorganism expressing the glucose-6-phosphate-isomerase, thereby converting the glucose-6-phosphate to fructose-6-phosphate.

Further, the preparation method of the present disclosure may further include, before the step of converting fructose-6-phosphate of the present disclosure to fructose-6-phosphate, a step of contacting glucose-6-phosphate with phosphoglucomutase, a microorganism expressing the phosphoglucomutase or a culture of a microorganism expressing the phosphoglucomutase, thereby converting the glucose-1-phosphate to glucose-6-phosphate.

Further, the preparation method of the present disclosure may further include, before the step of converting glucose-6-phosphate of the present disclosure to fructose-6-phosphate, a step of contacting glucose with a glucose kinase, a microorganisms expressing the glucose kinase or a culture of a microorganism expressing the glucose kinase, and phosphate, thereby converting the glucose to glucose-6-phosphate.

The preparation method of the present disclosure may further include, before the step of converting glucose-1-phosphate of the present disclosure to glucose-6-phosphate, a step of contacting starch, maltodextrin, sucrose or a combination thereof with α-glucan phosphorylase, starch phosphorylase, maltodextrin phosphorylase or sucrose phosphorylase; a microorganism expressing the phosphorylase; or a culture of a microorganism expressing the phosphorylase, and phosphate, thereby converting the starch, maltodextrin, sucrose or a combination thereof to glucose-1-phosphate.

The preparation method of the present disclosure may further include, before the step of converting the glucose of the present disclosure to glucose-6-phosphate, a step of contacting starch, maltodextrin, sucrose or a combination thereof with α-amylase, pullulanase, glucoamylase, sucrose or isoamylase; a microorganism expressing the amylase, pullulanase or sucrose; or a culture of microorganisms expressing the amylase, pullulanase or sucrase, thereby converting the starch, maltodextrin, sucrose or a combination thereof to glucose.

The preparation method of the present disclosure may further include a step of contacting glucose with 4-α-glucanotransferase, a microorganism expressing the 4-α-glucanotransferase or a culture of a microorganism expressing the 4-α-glucanotransferase, thereby converting the glucose to starch, maltodextrin or sucrose.

The allulose produced according the preparation method can be usefully used by adding it to functional foods and pharmaceuticals.

### ADVANTAGEOUS EFFECTS

The fructose-6-phosphate 3-epimerase according to the present disclosure satisfies at least one of the properties of high enzyme conversion rate, acidic or neutral reaction pH conditions, and high thermal stability, and thus, can be usefully used for the production of ketohexose using fructose-6-phosphate 3-epimerase, on an industrial scale.

### BRIEF DESCRIPTION OF THE DRAWINGS

FIG. 1 is an electrophoresis photograph confirming the expression and purification of fructose-6-phosphate 3-epimerase protein according to an embodiment of the present disclosure;

FIG. 2 shows the results of BIO-LC analysis of fructose-6-phosphate 3-epimerase protein according to an embodiment of the present disclosure;

FIG. 3 shows the results of LC analysis after dephosphorylating the phosphorylated saccharide in the reaction solution through the enzymatic reaction of allulose-6-phosphate phosphatase according to an example of the present disclosure;

FIG. 4 is a graph showing the results of analyzing the temperature characteristics of fructose-6-phosphate 3-epimerase protein according to an embodiment of the present disclosure;

FIG. 5 is a graph showing the results of analyzing the pH characteristics of fructose-6-phosphate 3-epimerase protein according to an embodiment of the present disclosure;

FIG. 6 is a graph showing the effect of metal ions of fructose-6-phosphate 3-epimerase protein according to an embodiment of the present disclosure; and

FIG. 7a and FIG. 7b is an HPLC analysis result of a reaction product obtained after performing allulose production using three types of conventionally known ribulose-phosphate 3-epimerase.

### DETAILED DESCRIPTION OF THE EMBODIMENTS

The present disclosure will be described in more detail with reference to the following examples, but the scope of the lights is not limited to the following examples.

### Example 1: Production of Fructose-6-Phosphate 3-Epimerase

Candidate enzymes expected to function as fructose-6-phosphate 3-epimerase were screened, and as the enzyme expected to show the best effect, polynucleotide (SEQ ID NO: 2) encoding the amino acid sequence (SEQ ID NO: 1) of the enzyme (ClFP3E) derived from Clostridium lundense DSM 17049 strain was obtained by requesting gene synthesis through IDT gene synthesis. A primer was designed based on the synthesized ClFP3E DNA sequence of SEQ ID NO: 2, and PCR was performed to amplify the nucleotide sequence of the gene. The forward and reverse primer sequences used for PCR amplification are as follows.

TABLE 1

| | | SEQ |
|---|---|---|
| | | ID |
| Primer name | Sequence (5′->3′) | NO: |
| pET21_CIRP3E_F | tatacatatgAAATACGGCTTCGCACC | 3 |
| pET21_CIRP3E_R | ggtgctcgagTTCTTTGTAGCTCAGGCTGTA | 4 |

The ClFP3E gene obtained in large quantities was introduced into the pET21a vector using restriction enzymes NdeI and XhoI to prepare pET21_ClFP3E, which was transformed into Escherichia coli ER2566 strain. Recombinant. E. coli for enzyme protein expression was obtained as colonies on an agar plate prepared in LB medium containing 50 μg/ml ampicillin.

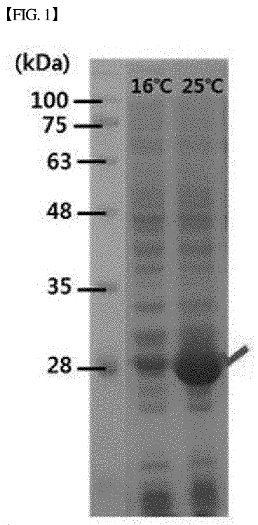

After seed culture in 4 ml LB medium, the main culture was performed in 100 ml LB medium. The culture conditions were incubated at 37° C. and 200 rpm until the absorbance value at 600 nm was 0.6, and then 0.1 mM IPTG was added thereto to induce expression of the target protein. After induction, the strain was cultured at 25° C. for about 16 hours, and then centrifuged to recover the cells. The recovered cells were suspended in a lysis buffer (50 mM sodium phosphate (pH 7.0) buffer, 300 mM NaCl, 10 mM imidazole), and the cells were disrupted using a beadbeater. The overexpression of the target protein ClFP3E from the cell disruption solution was confirmed by SDS-PAGE gel analysis. The results of overexpression analysis of the target protein ClFP3E are shown in FIG. 1. The molecular weight of ClFP3E confirmed by SDS-PAGE gel analysis was about 28 KDa.

Additionally, after removing the cell pellet, only the cell supernatant was obtained and bound to a Ni-NTA column (Ni-NTA superflow, Qiagen), and then proteins not bound to the column were removed with a washing buffer (50 mM sodium phosphate (pH 7.0) buffer, 300 mM NaCl, 20 mM imidazole). As a final step, the protein of interest was eluted with an elution buffer (50 mM sodium phosphate (pH 7.0) buffer, 300 mM NaCl, 200 mM imidazole). The finally secured protein was converted to 501 mM sodium phosphate buffer (pH 7.0) and stored for subsequent use.

### Example 2: Evaluation of the Conversion Activity of Enzymes

0.1 mg/ml of the purified ClFP3E enzyme obtained in Example 1 was added to a solution in which 20 g/L of fructose-6-phosphate was dissolved in 50 mM sodium phosphate (pH 7.0) buffer, and the enzymatic reaction was performed at 50° C.

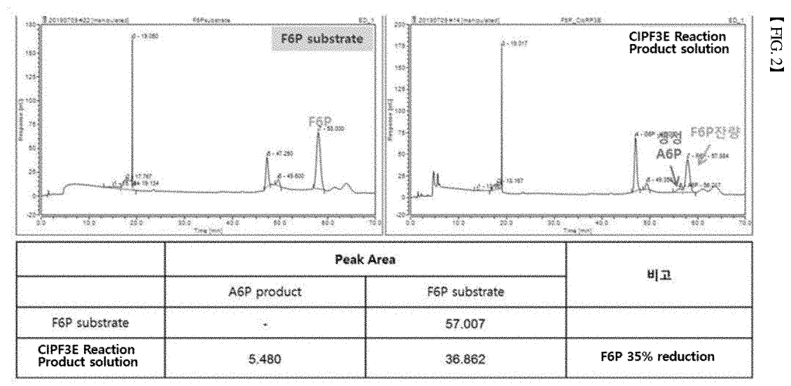

The analysis of the enzymatic reaction solution confirmed a newly produced substance as compared with the substrate through Bio-LC analysis. However, since the allulose-6-phosphate standard did not exist, accurate confirmation was not possible, and thus, after further treatment with a phosphatase enzyme of allulose-6-phosphate (A6PP), the resulting allulose was finally confirmed. Bio-LC analysis confirmed that during the C1FP3E enzyme reaction, a decrease in F6P and a new peak thought to be A6P were generated. The results of the Bio-LC analysis are shown in FIG. 2 below.

The result of LC analysis after dephosphorylating the phosphorylated saccharide in the reaction solution through the A6PP enzymatic reaction is as follows. Analysis was performed at a temperature of 80° C. and a flow rate of 0.6 ml/min using Aminex HPX-87C column, and the results are shown in FIG. 3. As a result of the analysis, fructose and allulose could be confirmed.

The final conversion rate of ClFP3E calculated by quantifying the amount of allulose produced, which is the final product of the reaction solution dephosphorylated by the Arlos-6-phosphate phosphatase (A6PP) enzymatic reaction, was calculated to be 34.3%. T The reaction product of this example is the final conversion of ClFP3E obtained by performing the enzymatic reaction for 16 hours, and the maximum conversion rate of allulose is calculated according to the following equation.

### Equation 1

Allulose maximum conversion rate (%)=allulose production amount (g/L)/fructose-6-phosphate input amount (g/L)*(allulose molecular weight/fructose-6-phosphate molecular weight)*100

### Example 3: Analysis of Temperature Characteristics of Enzymes

To determine the effect of temperature on ClFP3E enzyme activity, 10 g/L of fructose-6-phosphate was dissolved in 50 mM sodium phosphate (pH 7.0) buffer, and then 0.01 mg/ml of ClFP3E purified protein was added thereto, and the reaction was performed at various temperature conditions between 40 and 80° C. for 5 minutes.

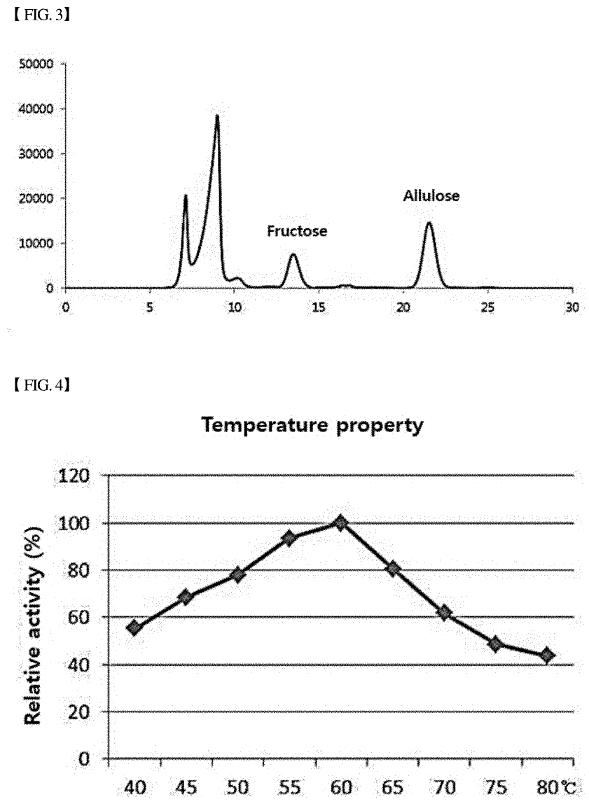

Then, the A6PP enzyme was added to dephosphorylate all reaction compositions, and then, the amount of allulose produced was quantitatively analyzed through HPLC analysis. The relative activity of the enzyme according to the reaction temperature is shown in FIG. 4 based on the activity at 60° C. Where the highest activity was measured.

As a result of the experiment, it was confirmed that the optimum temperature condition for ClFP3E was confirmed to be 60° C., and the activity was 50% or more of the maximum enzyme activity in a wide temperature condition range of 40 to 70° C.

### Example 4: Analysis of pH Characteristics of Enzymes

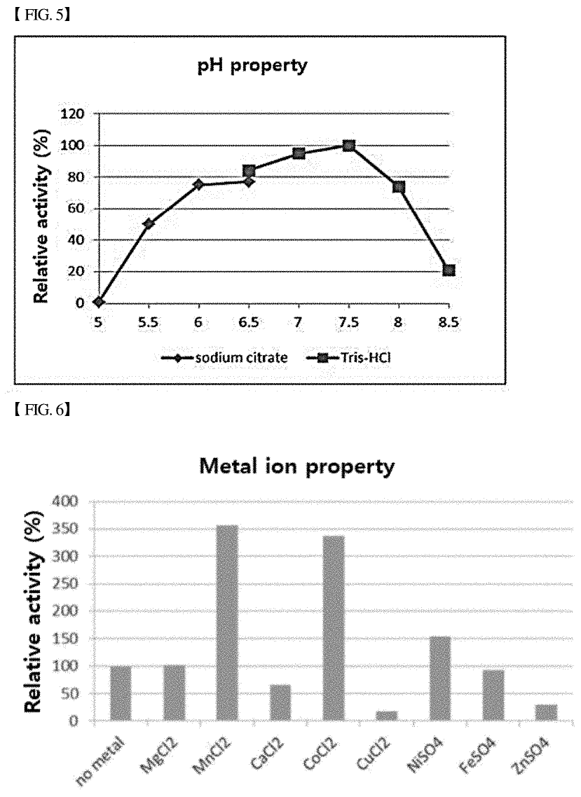

To confirm the effect of pH on ClFP3E enzyme activity, 10 of fructose-6-phosphate was dissolved in a buffer of pH 5.0˜8.5(pH 5.0˜6.5, sodium citrate/pH 6.5˜8.5, Tris -HCI), 0.01 mg/ml of purified ClFP3E protein was added thereto, and the enzymatic reaction was performed at a temperature of 60° C. for 5 minutes. Then, A6PP enzyme was added to dephosphorylate all reaction compositions, and then allulose production amount was quantitatively analyzed through HPLC analysis. The result of the enzyme. activity according to the reaction pH is shown in FIG. 5 as a relative activity based on pH 7.5, which had the best activity.

As a result of the experiment, it was possible to confirm the maximum activity at pH 7.0 to 7.5, and it was confirmed that it had 80% or more of the maximum enzyme activity in the range of pH 6.0 to 8.0.

### Example 5: Analysis of the Effect of Metal Ions on Enzymes

In order to confirm the activity of ClFP3E according to the type of metal ion added during the reaction, 10 g/L of fructose-6-phosphate was dissolved in 50 mM sodium phosphate (pH 7.0) buffer, 5 mM of each metal ion (MgCl₂, MnCl₂, CaCl₂, CoCl₂, CuCl₂, NiSO₄, FeSO₄, ZeSO₄) was added. 0.01 mg/ml of ClFP3E purified protein was added to the reaction buffer containing each metal ion, and the reaction was carried out at a temperature of 60° C. for 5 minutes.

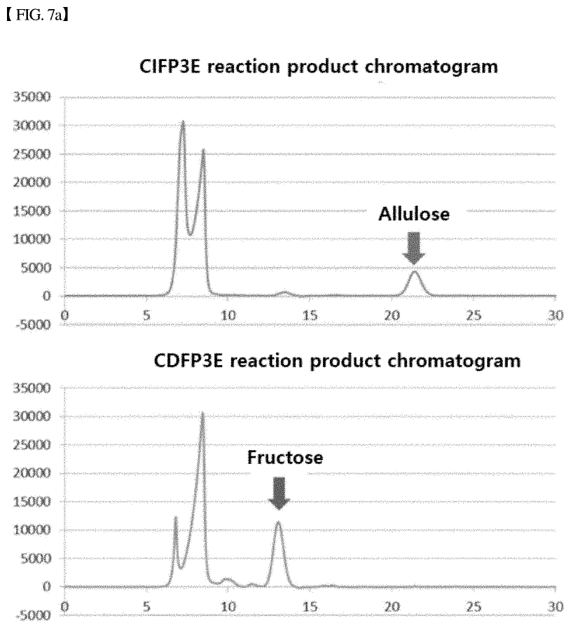

Then, the A6PP enzyme was added to dephosphorylate all reaction compositions, and then, the amount of allulose produced was quantitatively analyzed through HPLC analysis. The relative activity of the enzyme depending on the type of the metal ion is shown in FIG. 6 based on the experimental group in which no metal ion was added.

As a result of the experiment, when MnCl₂ and CoCl₂ were added, it was confirmed that the activity was three times higher than the condition in which no metal ions were added. it was found that the ClFP3E enzyme is an enzyme using manganese and cobalt as cofactors. Also, the activity was increased even by nickel. It was CaCl₂, CuCl₂, FeSO₄, ZeSO₄ that decreased the enzyme activity.

### Comparative Example 1: Analysis of Allulose Production Using Ribulose-Phosphate 3-Epimerase

Polynucleotides of Ruminococcus sp. AF14-10-derived ribulose-phosphate 3-epimerase (RuFP3E: amino acid sequence of SEQ ID NO: 5 and nucleic acid sequence of SEQ ID NO: 6), Clostridium sp. DU-VDT-derived ribulose-phosphate 3-epimerase (CDFME: amino acid. sequence of SEQ ID NO: 7 and nucleic acid sequence of SEQ ID NO: 8), Paenibacillus kribbensis-derived ribulose-phosphate 3-epimerase (PkFP3E: amino acid sequence of SEQ ID NO: 9 and nucleic acid sequence of SEQ ID NO: 10) were obtained by requesting gene synthesis.

As a result of analyzing the amino acid sequence homology to the amino acid sequence (SEQ ID NO: 1) of fructose-6-phosphate 3-epimerase (ClFP3E) derived from the Clostridium lundense DSM 17049 strain, the amino acid sequence identity to RuFP3E having the amino acid sequence of SEQ ID NO: 5 was 59.05%, the amino acid sequence identity to CDFP3E having the amino acid sequence of SEQ ID NO: 7 was 63%, and the amino acid sequence identity to PkFP3E having the amino acid sequence of SEQ ID NO: 9 was 60.96%.

Based on the synthesized ClFP3E DNA sequence of SEQ ID NO: 2, a large amount of genes were obtained in substantially the same method as in Example 1. In substantially the same method as in Example 1, the obtained gene was introduced into and expressed in E. Coli and then the protein of interest was eluted. Finally, the obtained protein was convened to 50 mM sodium phosphate buffer (pH 7.0) and stored for subsequent use.

In order to analyze the conversion activity of fructose 6-phosphate to allulose 6-phosphate, an enzymatic reaction using fructose-6-phosphate as a substrate was performed using the obtained enzyme in the same method as in Example 2. After the enzymatic reaction, a dephosphorylation reaction using an allulose-6-phosphate phosphatase (A6PP) enzyme was performed, and then, the amount of produced allulose was measured for the reaction product. solution using High-Performance Liquid Chromatography (HPLC).

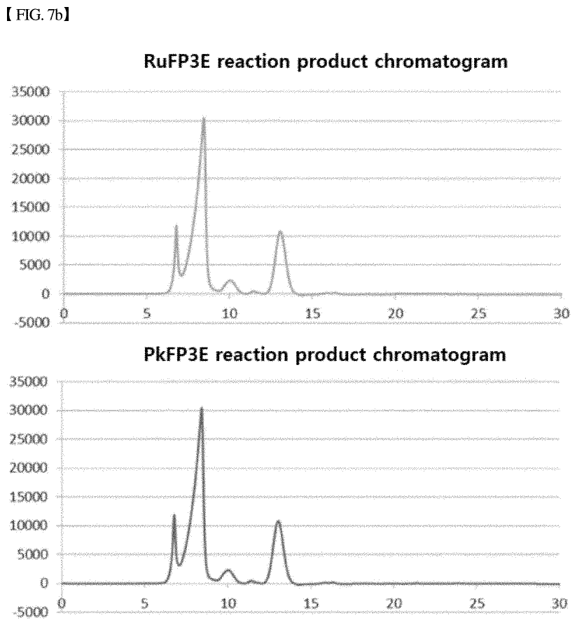

As for HPLC analysis conditions, RID (Refractive Index Detector Agilent 1260 RID) of HPLC (Agilent, USA) equipped with an Aminex HPX-87C column (BIO-RAD) was used. Water was used as the mobile phase solvent, and the temperature was 80° C. and the flow rate was 0.6 ml/min. The results of the HPLE analysis are shown in FIG. 7a and 7b. The reaction product of this example was obtained by performing the enzymatic reaction for 4 hours to obtain the allulose conversion rate of the enzyme. As an experiment for screening the FP3E candidate enzyme, the reaction solution in which the reaction was stopped at the intermediate stage of allulose production was analyzed. In FIG. 7a and FIG. 7b, the allulose conversion rate of ClFP3E was 16%, and the reaction proceeded to about 50% level of the maximum conversion rate of Example 2. In this Example, the allulose production ratio of total reaction products was 75%. The alullose production rate is calculated by the following Equation.

Allulose production ratio (% by weight)=production amount/(fructose production amount+allulose production amount)*100

As shown in FIG. 7a and FIG. 7b, no allulose peak was confirmed in other candidate enzymes except for the ClFP3E enzyme of Example 1. Therefore, because other candidate enzymes other than the ClFP3E enzyme had no activity against F6P, it was confirmed that when phosphatase was treated, only fructose of a dephosphorylated from produced from F6P as an initial substrate of the reaction step, was generated.

1.
An epimerase protein of fructose-6-phosphate which has at least 70% of amino acid sequence identity to the amino acid sequence of SEQ ID NO: 1 and converts fructose-6-phosphate to allulose-6-phosphate.

2.
The epimerase protein according to claim 1, wherein the enzyme is encoded by a nucleotide sequence having at least 80% of nucleotide sequence identity to the nucleotide sequence of SEQ ID NO: 2.

3.
The epimerase protein according to claim 1, wherein the enzyme is derived from Clostridium lundense, and has an enzyme reaction temperature of 40 to 70° C. and an enzyme reaction pH of pH 6 to 8.

4.
The epimerase protein according to claim 1, wherein the enzyme is an incrased activity by a manganese ion, a cobalt ion, or a nickel ion.

5.
A composition for producing allulose, comprising at least one selected from the group consisting of fructose-6-phosphate epimerase protein as set forth claim 1, a microorganism expressing the enzyme, a transgenic microorganism expressing the enzyme protein, a microbial cell of the microorganism, cell lysate of the micribial cell of the microorganism, a culture of the microorganism, a culture supernatant of the microorganism, a concentrate of the culture supernatant of the microorganisms and their powders.

6.
The composition for producing allulose according to claim 5, wherein the composition further comprises a phosphatase enzyme of allulose 6-phosphate, a microorganism expressing the same, or a culture of the microorganism.

7.
The composition for producing allulose according to claim 5, wherein the composition further comprises one or more metal ions selected from the group consisting of manganese ion, cobalt ion and nickel ion.

8.
The composition for producing allulose according to claim 5, wherein the composition further comprises an isomerase converting glucose-6-phosphate to fructose-6-phosphate, a microorganism expressing the same, or a culture of the microorganism.

9.
The composition for producing allulose according to claim 5, wherein the composition further comprises:
(a) (i) starch, maltodextrin, sucrose, or a combination thereof; (ii) phosphate; (iii) allulose-6-phosphate phosphase; (iv) glucose-6-phosphate isomerase; (v) phosphoglucomutase or glucose phosphorylase; and (vi) α-glucanophosphorylase, starch phosphorylase, maltodextrin phosphorylase, sucrose phosphorylase, α-amylase, pullulanase, isoamylase, glucoamylase or sucrase; or
(b) a microorganism expressing the enzyme of item (a) or a culture of the microorganism, and
wherein the composition further comprises a phosphatase enzyme of allulose 6-phosphate.

10.
A method for producing allulose, comprising a step of converting fructose-6-phosphate to allulose-6-phosphate using the epimerase of fructose-6-phosphate as set forth in claim 1.

11.
The method according to claim 10, which further comprises a step of converting allulose-6-phosphate to allulose by contacting the allulose-6-phosphate with phytase, a microorganism expressing the same, or a culture of the microorganism.

12.
The method according to claim 10, which the step of converting fructose-6-phosphate to allulose-6-phosphate is performed at a reaction temperature of 40 to 70° C. and a reaction pH of pH 6 to 8.

13.
The method according to claim 10, which further comprises a step of preparing fructose-6-phosphate from fructose or a fructose-containing material using hexokinase.


SEQUENCE LISTING (10 sequences)

SEQ ID NO: 1 - protein, 227 aa, Artificial Sequence
  Fructose 6-phosphate 3-epimerase
MKYGFAPSLMCMNILKLEEQIKILNKKADFYHVDIMDGHYVKNITLSPFFIQQIRPLATIPIDAHLMVENPNDFLDELKKAGTDYVSLHVETINKDVFRITNKIKSLGMKVGVVLNPATPIDEIKHYINRLDKITVMTVDPGFAGQAFIPEMLDKIRELKEIKEKEGYKYIIEIDGSCNEKTFAQLKEAGAEVFVVGSSGLFNKDEDLEKAWEMMMSDFNYSLSYKE

SEQ ID NO: 2 - DNA, 684 bp, Artificial Sequence
  Polynucleotide sequence for fructose 6-phosphate 3-epimerase
atgaaatacggcttcgcaccgtccctgatgtgcatgaacatcctgaaactggaagaacaaatcaaaattctgaacaaaaaagcggatttctaccacgtagacattatggatgggcactatgttaaaaacatcacgctgtctccgttctttatccaacagatccgcccgctggcgaccattccaatcgacgcgcatctgatggtcgagaacccgaacgactttctggacgaactgaaaaaagctggtactgactacgtgtctctgcacgtagaaaccattaacaaagatgttttccgcatcacgaacaaaatcaaatcgttaggtatgaaagttggcgtggttctgaaccctgcgactccgatcgacgaaatcaaacattacatcaaccgtctggacaaaatcactgttatgactgttgaccctggtttcgctggccaagcgttcatcccggaaatgctggacaaaatccgtgaactcaaggaaatcaaagaaaaagagggttacaaatacattattgaaatcgatggcagctgcaacgaaaagactttcgcccaactgaaagaagcgggtgcggaagttttcgtagtcggctctagcggcctgtttaacaaagatgaagacctggaaaaagcctgggaaatgatgatgtccgacttcaattacagcctgagctacaaagaatag

SEQ ID NO: 3 - DNA, 27 bp, Artificial Sequence
  Primer pET21_ClRP3E_F
tatacatatgaaatacggcttcgcacc

SEQ ID NO: 4 - DNA, 31 bp, Artificial Sequence
  Primer pET21_ClRP3E_R
ggtgctcgagttctttgtagctcaggctgta

SEQ ID NO: 5 - protein, 225 aa, Artificial Sequence
  Ribulose-phosphate 3-epimerase (RuFP3E)
MKVEFSPSLMCMDFLKMEEQFNILNDKVDMYHIDIMDGHFCKNITLSPDLVKTFSKVAKKPMDVHLMTTNPTDWIEKVAAAGAEYISPHAETINGDAFRVMNMIKSLGCKTGVVLNPATPLSYVKHYLNRIDLLTIMTVDVGFAGQPFIEEMLDKIKEAKELKEKYGYTYKIQIDGSCNPKTYKRMYDAGAEVLIVGSSGLFGLNEDLNKACDQMFEDFNRAIAE

SEQ ID NO: 6 - DNA, 678 bp, Artificial Sequence
  Polynucleotide seqence for ribulose-phosphate 3-epimerase (RuFP3E)
atgaaagtagaattttctccgtccctgatgtgcatggacttcctgaagatggaagaacagttcaacattctgaatgacaaagtggacatgtatcatatcgacatcatggacggccatttctgcaagaacatcaccctgtccccggatctggttaaaaccttttctaaggtagcgaaaaaaccgatggatgtgcacttgatgaccaccaacccgaccgattggatcgaaaaagttgccgcggcgggcgctgagtacattagtccgcacgccgaaaccattaatggtgatgctttccgcgtgatgaacatgatcaagagcctgggctgcaaaaccggcgtagtactgaacccggccaccccgctgtcctatgttaaacactatctgaaccgcattgacctgctgaccattatgactgtagacgttggtttcgcgggccagccgttcatcgaggaaatgctggataaaatcaaagaagctaaagaactgaaagagaaatatggttacacctacaagatccagatcgacggctcttgcaacccgaaaacctataagcgtatgtacgacgcaggcgctgaagttctgattgtgggttctagcggtctgttcggtctgaacgaagatctgaacaaggcatgtgatcagatgttcgaagacttcaaccgtgcgatcgcagaataa

SEQ ID NO: 7 - protein, 225 aa, Artificial Sequence
  Ribulose-phosphate 3-epimerase (CDFP3E)
MKPMFAPSLMCANFLDLKNQIEILNERADIFHVDIMDGHYVKNFSLSPAMMEQLKTITKIPMDAHLMVENPADFLEGIAKAGATYISPHAETINKDAFRIMRTIKALGCKTGVVLNPATPVEYIKHYLGMLDKITILTVDAGFAGQTFIEEMLDKIEEVKRLREENGYSYLIEVDGSCNEKTFKKLAEAGTEVFIVGSSGLFNLDADLKVSWDKMMNMFNKCINN

SEQ ID NO: 8 - DNA, 678 bp, Artificial Sequence
  Polynucleotide sequence for ribulose-phosphate 3-epimerase (CDFP3E)
atgaagccgatgtttgctccgtctctgatgtgcgctaactttctcgatctcaaaaaccaaatcgaaatcttgaacgaacgcgctgacatcttccatgtagacatcatggatggccattacgtaaagaatttttctctctctccggcgatgatggaacagcttaaaaccattactaaaatcccgatggacgcccacctgatggtggaaaacccggcggatttcctggaaggaatcgcaaaagcgggcgcgacctacatcagccctcacgccgaaaccattaacaaagatgcgtttcgtatcatgcgcaccatcaaagctctgggctgtaaaactggtgttgtgctgaatccggccactccggtagagtacattaaacactatctgggcatgctggacaaaattaccatcctgactgttgacgcaggtttcgcgggtcagacctttatcgaagagatgctggataaaattgaagaagttaaacgtctgcgtgaagaaaacggttactcgtatttaattgaggtcgacggttcctgcaacgagaagactttcaaaaaattagcggaagctggtaccgaagttttcattgttggctctagcggcctgttcaacctggacgcggacctgaaagtgagctgggataaaatgatgaacatgttcaataaatgtatcaacaactaa

SEQ ID NO: 9 - protein, 228 aa, Artificial Sequence
  Ribulose-phosphate 3-epimerase (PkFP3E)
MKKHLFSPSLMCMNLMDIENQLKVLNQRADMVHIDIMDGHYVKNITLSPYFMEQIRESLTIPMDVHLMVEKPSEFIKMIIDAGATYISPHAEVINSEAFRMIDTIKSAGCKAGIALNPATPLSHIQHYIHHLDKITFMSVDPGFAGQKFIPEVLDKIREARALKEKHGYNYLIEIDGSCNEKTFKILEEAGVEVFIVGTSGLFNLDSDLNKAWDQMMEHFNSQVNAEV

SEQ ID NO: 10 - DNA, 687 bp, Artificial Sequence
  Polynucleotide sequence for ribulose-phosphate 3-epimerase (PkFP3E)
atgaaaaaacacctgttcagcccgtctctgatgtgtatgaatctgatggacatcgaaaaccagctgaaagtactgaatcagcgcgcagacatggtacacattgatatcatggatggccattatgttaaaaacattaccctgagcccgtacttcatggaacagattcgcgaatcgctgactatccctatggatgttcatctgatggttgaaaaaccgagcgaattcattaaaatgattatcgatgcgggtgccacctacattagccctcatgctgaagttatcaactctgaagcgtttcgtatgatcgacaccatcaaatccgcgggttgcaaagctggtattgcactgaacccggcaaccccgctgtctcacatccaacattacatccatcatctcgacaagatcacctttatgtccgttgatcctggtttcgctgggcagaaattcatccctgaagtcctggataagattcgtgaagctcgtgcactgaaagaaaaacacggctacaactacttaatcgaaatcgatggttcttgcaatgagaaaacctttaagatcctcgaagaagcaggtgtagaagttttcatcgttggtacttcgggactgttcaacctggatagcgacctgaacaaggcttgggaccagatgatggaacatttcaactctcaggtcaacgcagaggtctga